(19) United States
(12) Patent Application Publication
(10) Pub. No.: US 2024/0041726 A1
(43) Pub. Date: Feb. 8, 2024

(54) FOAMABLE COMPOSITION, USE THEREOF, AND PROCESS FOR ITS PREPARATION

(71) Applicant: CHATTEM, INC., Bridgewater, NJ (US)

(72) Inventors: David SHARP, Chattanooga, TN (US); Duncan COFFEY, Chattanooga, TN (US)

(21) Appl. No.: 18/031,932

(22) PCT Filed: Oct. 22, 2021
(86) PCT No.: PCT/US2021/056159

§ 371 Date: Apr. 14, 2023

Related U.S. Application Data
(60) Provisional application No. 63/104,749, filed on Oct. 23, 2020.

(51) Int. Cl. A61K 8/34; A61K 8/04; A61Q 19/00

(52) U.S. Cl. CPC A61K 8/34; A61K 8/046; A61Q 19/00; A61K 2800/24; A61K 2800/596; A61K 2800/80

(57) ABSTRACT

Disclosed herein is a foamable composition and a foam, its use, and process for its preparation. The foamable composition comprises: short-chain alcohol(s); a surfactant; a sensation modifying agent; water; and optionally an additive and/or second acceptable surfactant. The composition provides physical stability, an improved foam quality, consumer/sensory benefits and is useful for topical application.

### CROSS-REFERENCE TO RELATED APPLICATIONS

This application claims priority to U.S. Provisional Application No. 63/104,749 (filed Oct. 23, 2020) the entire contents of which is incorporated by reference herein.

### FIELD OF THE INVENTION

Disclosed herein are a foamable composition, a foam, its use, and process for its preparation. The foamable composition comprises short-chain alcohol(s); a surfactant; a sensation modifying agent; water; a second surfactant, and optionally, an additive. The composition disclosed herein provides physical stability, an improved foam quality, consumer/sensory benefits and is useful for topical application.

### BACKGROUND OF THE INVENTION

Compositions of various types comprise components or agents which provide a sensation to the skin. The sensation may be a warming, cooling, relaxation, stimulation or refreshing feeling or a combination of two or more feelings. The sensation or feeling can be physically or chemically mediated. However, maintaining stability and good foam quality while increasing the amount of sensation modifying agents is challenging.

Foams are considered very convenient vehicles for topical delivery of active agents. There are several types of topical foams, including aqueous foams, hydroalcoholic foams, aerosol foams, emulsion-based foams, and oleaginous foams, among others.

Hydroalcoholic foam compositions comprise short-chain alcohols, preferably C₁-C₆ alkylalcohol such as ethanol, propanol, isopropanol, butanol, iso-butanol, t-butanol and pentanol; one or more surfactants such as non-ionic, anionic, cationic, zwitterionic, amphoteric and amphiphilic surfactants; and additives, such as fatty alcohols and polymers.

Surfactants are known as essential ingredients in foam compositions because of their amphiphilic properties by their ability to reduce surface tension and allowing bubbles to form without collapsing.

However, foams are complicated systems which do not form under all circumstances. Changes in foam composition, such as by the addition of active ingredients (e.g. sensation modifying agents), frequently destabilize the foam.

US 2013/0011490 discloses a foamable composition containing more than 40.0% v/v of C₁-C₄ alcohol, or mixtures thereof, at least 0.01% of an effective physiologically acceptable silicone-based surfactant for wetting and foaming, and water. Said document discloses that to achieve a commercially suitable composition, one of the approaches is to reduce the amount of silicone-based surfactant used while using the assistance of other ingredients such as secondary surfactants, emulsifiers, foam stabilizers, fragrances, as well ingredients employed in cosmetics, aerosols, toiletries, personal care, among others. US '490 discloses that the silicone-based surfactant may be a bis-PEG-[10-20] dimethicone, a 3-(3-Hydroxypropyl)-heptamethyltrisiloxane, an ethoxylated silicone-based surfactant, a bis-PEG/PPG 18/6 dimethicone, a Polyether-modified polysiloxane or a polysiloxane betaine, or mixtures thereof.

JP2013063939A discloses an aerosol foaming composition that is formed from a stock solution containing water, menthol, a surfactant, and a propellant. In the stock solution, the % weight contents are: menthol 0.1-1.0%, surfactant 15.05-21.50%; and a foam-like substance having stringiness is formed upon discharge.

U.S. Pat. No. 9,320,705 discloses foamable compositions containing: (a) a foamable carrier; (b) at least one sensation modifying agent, and (c) a propellant at a concentration of about 3% to about 45% by weight of the total composition. The foamable carrier can be an emollient emulsion, a foamable gel or a substantially non aqueous foamable vehicle. Example 2 of US '705 is directed to an emollient emulsion foamable composition containing 10% of menthol as sensation modifying agent, 15% of ethanol, 2.31% of PEG 40 stearate as stabilizing emulsifier, 10% of avocado oil as emollient, several other additives such as co-emulsifiers and foam stabilizer, emollients, stabilizing polymers, and water. Said document discloses that ethanol is used as a penetration enhancer, and that ethanol is not essential and may be omitted or replaced by other penetrating agents such as transcutol.

Known related compositions include the product Biofreeze®, which is available on the market and contains 7% of menthol, denatured alcohol, bis-PEG-10 dimethicone and PEG-12 dimethicone as surfactants. However, such composition has technical limitations, such as lack of ability to create foam with sensation modifying agent at higher percentages.

There remains a need for improved, easy to use, stable and/or non-irritating topical foam compositions containing with high sensation modifying agent. Compositions disclosed herein solve these technical problems, provides desirable properties to the skin (e.g. non-greasy, non-tacky, good skin feel, no irritation), and remains stable whilst enabling the use of a variety of and/or high amounts of sensation modifying agents.

### SUMMARY OF THE INVENTION

Disclosed herein are a foamable composition and a foam, its use, and a process for its preparation. The compositions are useful for topical application.

In one embodiment, disclosed herein is a foamable composition comprising:
(i) a short-chain alcohol or mixtures thereof;
(ii) a surfactant;
(iii) 7.5% weight or more of at least one sensation modifying agent;
(iv) water;
(v) a second surfactant; and optionally,
(vi) an additive.

The compositions disclosed herein provide a foam which is useful for topical application onto the skin.

In another embodiment, disclosed herein is a process for the preparation of foaming compositions.

### BRIEF DESCRIPTION OF THE FIGURES

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1A-C show different foam qualities upon testing different compositions. FIG. 1A shows a good quality foam; FIG. 1B shows a non-uniform foam; and FIG. 1C shows a poor foam.

### DETAILED DESCRIPTION OF THE INVENTION

The foamable compositions disclosed herein are useful for topical application. In certain embodiments, the composition comprises:
(i) a short-chain alcohol or mixtures thereof;
(ii) a surfactant;
(iii) 7.5% weight or more of at least one sensation modifying agent;
(iv) water; and, optionally,
(v) an additive and/or a second surfactant.

Multiple foaming compositions were embodied and tested in different sets of experiments described in the present patent application. For the purposes of this patent application, all % are by weight, except when stated on the contrary.

The term “about” as used herein can allow for a degree of variability in a value or range, for example, within 10%, within 5%, or within 1% of a stated value or of a stated limit of a range.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The short-chain alcohol in this patent application can also be a specially denatured alcohol, or SD-Alcohol. A specially denatured alcohol is a combination of ethanol and another chemical substance, e.g., ethyl acetate in SDA 29, 35, and 35A, or SDA 40B, 40C, which contains tert-butyl alcohol and denatonium benzoate. According to some further embodiments, the short-chain alcohol is selected from C₁-C₆ alkyl alcohol, such as ethanol, propanol, isopropanol, butanol, isobutanol, t-butanol, and pentanol.

In one embodiment, the short-chain alcohol is present in amount between 25% to 80%; between about 30% to about 75%; between about 35% to about 65%; between about 40% to about 60%; between about 35% to about 80%; between 45% to about 65% by weight of the total composition. In one embodiment, the short-chain alcohol is present in amount of about 50%. In another embodiment, the short-chain alcohol is present in an amount greater than 35% by weight of the total composition.

According to some embodiments, the surfactant and the second surfactant are selected from: sodium lauryl sulfate, ammonium lauryl sulfate, cocamidopropyl betaine, sodium lauryl gl. carb (and) lauryl glucoside, PEG-40 glyceryl cocoate & sodium coceth sulfate, capryly/capryl glucoside, sodium lauroyl sarcosinate, sodium lauroyl oat amino acids, sodium cocoyl apple amino acids, C15-19 alkane, decyl glucoside & cocamidopropyl betaine, decyl glucoside, sodium lauroamphoacetate, PEG-8 PG coco-glucoside dimethicone, hydoxypropyl bis-hydroxyethyldimonium chloride, cocamidopropyl hydroxysultaine, sorbitan oleate decylglucoside crosspolymer, bis-stearyl dimethicone, PEG-8 dimethicone, bis-PEG-8 dimethicone, PEG-12 dimethicone, bis-PEG-12 dimethicone, bis-PEG-10 dimethicone, bis-PEG/PPG-14/14 dimethicone, or PEG/PPG-18/18 dimethicone.

In one embodiment, the surfactant is a dimethicone-based surfactant. In another embodiment, the surfactant is a (PEG)ₓ dimethicone, wherein x is from about 6 to about 20. In another embodiment, the surfactant is Bis-PEG [y], wherein [y] means all the Bis-PEG compounds have y repeating oxyethylene groups.

In one embodiment, the dimethicone-based surfactant is selected from: PEG-8 PG coco-glucoside dimethicone, bis-stearyl dimethicone, PEG-8 dimethicone, PEG-12 dimethicone, bis-PEG-12 dimethicone, bis-PEG-10 dimethicone, bis-PEG-8 dimethicone, bis-PEG/PPG-14/14 dimethicone, and PEG/PPG-18/18 dimethicone. In one embodiment, the dimethicone-based surfactant is selected from the group consisting of bis-PEG-12 dimethicone, bis-PEG-10 dimethicone, and bis-PEG-8 dimethicone.

In one embodiment, the dimethicone-based surfactant is present in an amount between about 0.5% to about 10.0%; between about 1.0% to about 10.0%, between about 1.5% to about 10.0%; between about 2.0% to about 10.0%, between about 2.5% to about 10.0%, between about 3.0% to about 10.0%, between about 3.5% to about 10.0%, between about 4.0% to about 10.0%, between about 5.0% to about 10.0%, between about 6.0% to about 10.0%, between about 0.5% to about 6.0%; between about 1.5% to about 6.0%; between about 1.5% to about 5.0%, between about 2.0% to about 6.0%; or between about 2.5% to about 10.0% by weight of the total composition.

According to some further embodiments, water is present in an amount of not more than about 40.0% by weight of the total composition. In one embodiment, water is present in an amount of between about 10.0 to about 30.0%, between 15.0 to about 35.0%, or between about 15.0 to about 25.0% by weight of the total composition.

In one embodiment, the sensation modifying agent is selected from the group of a cooling agent; a warming agent; a relaxing or soothing agent; stimulating or refreshing agent; or mixtures thereof.

According to some embodiments, the sensation modifying agent includes capsaicin and/or methyl salicylate.

According to some further embodiments, the cooling agent is selected from camphor or a camphor derivative, menthol, an isomer of menthol, a menthol derivative, 4-methyl-3-(1-pyrrolidinyl)-2[5H]-furanone, WS-23, Icilin, Icilin Unilever Analog, 5-methyl-4-(1-pyrrolidinyl)-3-[2H]-furanone, 4,5-dimethyl-3-(1-pyrrolidinyl)-2[5H]-furanone, isopulegol, 3-(l-menthoxy)propane-1,2-diol, 3-(l-menthoxy)-2-methylpropane-1,2-diol, p-menthane-2,3-diol, p-menthane-3,8-diol, 6-isopropyl-9-methyl-1,4-dioxas-piro[4,5]decane-2-methanol, menthyl succinate and its alkaline earth metal salts, trimethylcyclohexanol, N-ethyl-2-isopropyl-5-methylcyclohexanecarb-oxamide, Japanese mint (Mentha arvensis) oil, peppermint oil, menthone, menthone glycerol ketal, menthyl lactate, 3-(l-menthoxy)ethan-1-ol, 3-(l-menthoxy)propan-1-ol, 3-(l-menthoxy)butan-1-ol, l-menthylacetic acid N-ethylamide, l-menthyl-4-hydroxypentanoate, l-menthyl-3-hydroxybutyrate, N,2,3-trimethyl-2-(1-methylethyl)-butanamide and spearmint oil.

According to some embodiments, the menthol derivative is selected from the group consisting of: menthol ethylene glycol carbonate, menthol propylene glycol carbonate, menthyl lactate, menthone glycerin acetal and 3-(l-menthoxy)-1,2-propanediol. In one embodiment, the cooling agent is menthol.

In one embodiment, the cooling agent is present in an amount of between about 7.5% and about 25% by weight of the total composition. In one embodiment, the cooling agent is present in an amount of between about 7.5% and about 8.5%, between about 7.5% and about 9.0%, between about 7.5% and about 10.5%, between about 9.0% and about 12.0%, between about 9.0% and about 13.0%, between about 9.0% and about 14.0%, between about 9.0% and about 15.0%, between about 9.0% and about 16.0%, between about 9.0% and about 17.0%, between about 10.0% and about 13.0%, between about 10.0% and about 14.0%, between about 10.0% and about 15.0%, between about 10.0% and about 16.0%, between about 10.0% and about 17.0%, or between about 10.0% and about 18.0%, by weight of the total composition. In one embodiment, the cooling agent is present in an amount of about 10.0% or about 16.0% by weight of the total composition.

According to some further embodiments, the additive is selected from the group comprising distearyldimonium chloride, ethoxydiglycol, PPG12/SMDI copolymer, PPG-benzyl ether myristate, B-LC/HDI/PEG-100 copolymer, polysorbate 60, cetyl dimethicone, acrylates/C10-30 alkyl acrylate crosspolymer, PEG-3 dioleoylamidoethylmonium, methyl gluceth-20, polyquaternium-67, PEG-14M, propylene glycol, cocamidopropyl betaine, PEG-12-dimethicone, dimethicone 20 cosmetic, dimethicone crosspolymer blend, glycerin 99% kosher usp, diisopropyl adipate, ethylhexylglycerin, isopropyl myristate, HPMC 4000, behentrimonium methosulfate. In one embodiment, the additive is propylene glycol.

In one embodiment, the additive is presented in an amount of less than 7.5% by weight of the total composition. In one embodiment, the additive is presented in an amount of between about 0.1% to about 10.0%, between about 0.5% to about 10.0%; between about 1.0% to about 10.0%, between about 0.5% to about 5.0%, between about 0.5% to about 6.0%, between about 0.5% to about 7.0%, between about 0.5% to about 8.0%, or between about 0.1% to about 10.0% by weight of the total composition. In one embodiment, the additive is present in an amount of about 2.5% by weight of the total composition.

In one embodiment, a foamable composition is described comprising:
(i) a short-chain alcohol or mixtures thereof, present in an amount of greater than 35.0% by weight of the total composition;
(ii) a surfactant, present in an amount of at least 0.1% by weight of the total composition;
(iii) 7.5% weight or more of menthol; and
(iv) water.

In one embodiment, a foamable composition as described above further comprises at least one of the following ingredients:
(i) an additive; and/or
(ii) a second surfactant, present in an amount of at least 0.1% by weight of the total composition.

In one embodiment, a foamable composition is described comprising:
(i) a short-chain alcohol or mixtures thereof, present in an amount of greater than 35.0% by weight of the total composition;
(ii) a surfactant, present in an amount of at least 0.1% by weight of the total composition;
(iii) 7.5% weight or more of menthol;
(iv) a second surfactant, present in an amount of at least 0.1% by weight of the total composition; and
(v) water.

In one embodiment, a foamable composition as described above further comprises an additive.

In one embodiment, a foamable composition as described above comprises:
(i) a short-chain alcohol or mixtures thereof, present in an amount of greater than 35.0% by weight of the total composition;
(ii) a dimethicone-based surfactant, present in an amount of at least 0.1% by weight of the total composition;
(iii) a second dimethicone-based surfactant, present in an amount of at least 0.1% by weight of the total composition;
(iv) 7.5% weight or more of menthol; and
(v) water.

In one embodiment, the foamable composition comprises:
(i) 35.0% to 80.0% of SD-alcohol;
(ii) 0.5% to 10.0% of a dimethicone surfactant;
(iii) 7.5% to 18.0% of menthol; and
(iv) water.

In one embodiment, the foamable composition comprises:
(i) 35.0% to 80.0% of SD-alcohol;
(ii) 0.5% to 10.0% of a dimethicone surfactant; (iii) 7.5% to 18.0% of menthol;
(iv) 0.5% to 6.0% of a second dimethicone surfactant; and
(v) water.

In one embodiment, the foamable composition comprises:
(i) 35.0% to 80.0% of ethanol;
(ii) 0.5% to 10.0% of a dimethicone surfactant;
(iii) 7.5% to 18.0% of menthol;
(iv) 0.5% to 6.0% of a second dimethicone surfactant; and
(v) water.

In one embodiment, the foamable composition comprises:
(i) 35.0% to 65.0% of SD-alcohol;
(ii) 3.0% to 10.0% of a dimethicone surfactant;
(iii) 7.5% to 16.0% of menthol; and
(iv) water.

In one embodiment, the foamable composition comprises:
(i) 35.0% to 65.0% of ethanol;
(ii) 3.0% to 10.0% of a dimethicone surfactant;
(iii) 7.5% to 16.0% of menthol; and
(iv) water.

In one embodiment, the foamable composition comprises:
(i) 35.0% to 65.0% of SD-alcohol;
(ii) 3.0% to 10.0% of a dimethicone surfactant;
(iii) 7.5% to 16.0% of menthol;
(iv) 0.5% to 6.0% of a second dimethicone surfactant; and
(v) water.

In one embodiment, the foamable composition comprises:
(i) 35.0% to 65.0% of ethanol;
(ii) 3.0% to 10.0% of a dimethicone surfactant;
(iii) 7.5% to 16.0% of menthol;
(iv) 0.5% to 6.0% of a second dimethicone surfactant; and
(v) water.

In one embodiment, a foamable composition as described above further comprises 0.5% to 5.0% of additive.

In one embodiment, the foamable composition comprises:
(i) 45.0% to 65.0% of SD-alcohol;
(ii) 1.0% to 6.0% of bis-PEG-12 dimethicone;
(iii) 2.0% to 6.0% of bis-PEG-8 dimethicone;
(iv) 7.5% to 16.0% of menthol;
(v) water; and, optionally,
(vi) 0.5% to 5.0% of an additive.

In one embodiment, the foamable composition comprises:
(i) 45.0% to 65.0% ethanol;
(ii) 1.0% to 6.0% of bis-PEG-12 dimethicone;
(iii) 2.0% to 6.0% of bis-PEG-8 dimethicone;
(iv) 7.5% to 16.0% of menthol;
(v) water; and, optionally,
(vi) 0.5% to 5.0% of an additive.

In one embodiment, the foamable composition comprises:
(i) 45.0% to 65.0% of SD-alcohol;
(ii) 1.5% to 4.0% of bis-PEG-12 dimethicone;
(iii) 3.5% to 6.0% of bis-PEG-8 dimethicone;
(iv) 7.5% to 16.0% of menthol;
(v) water; and
(vi) 0.5% to 5.0% of an additive.

In one embodiment, the foamable composition comprises:
(i) 45.0% to 65.0% of ethanol;
(ii) 1.5% to 4.0% of bis-PEG-12 dimethicone;
(iii) 3.5% to 6.0% of bis-PEG-8 dimethicone;
(iv) 7.5% to 16.0% of menthol;
(v) water; and
(vi) 0.5% to 5.0% of an additive.

In one embodiment, the foamable composition comprises:
(i) 45.0% to 65.0% of ethanol;
(ii) 1.5% to 4.0% of bis-PEG-12 dimethicone;
(iii) 3.5% to 6.0% of bis-PEG-8 dimethicone;
(iv) 7.5% to 16.0% of menthol;
(v) water; and
(vi) 0.5% to 5.0% of an additive.
   In this embodiment, the composition is mixed with air through a fine mesh screen at low pressure to form a foam.

In any of the embodiments above, the SD-alcohol or ethanol may be replaced with any of the other short-chain alcohol or mixture thereof disclosed herein.

The disclosure further provides a method for relieving pain comprising: applying to a person's skin a foam composition which comprises
a) air mixed with
b) a composition comprising
   (i) a short-chain alcohol or mixtures thereof;
   (ii) a surfactant;
   (iii) 7.5% weight or more of at least one sensation modifying agent;
   (iv) water;
   (v) optionally, an additive; and
   (vi) optionally, a second surfactant.
      In one embodiment, the composition used in the method contains no additive or surfactant. In another embodiment, the composition contains an additive or a second surfactant. In an alternate embodiment, the composition contains an additive and a second surfactant.

The disclosure further provides a method for producing, and applying to a person's skin, a foam composition comprising: a) combining a short-chain alcohol or mixtures thereof, a surfactant, 7.5% weight or more of at least one sensation modifying agent, optionally, an additive and optionally, a second surfactant and storing said composition in an unpressurized dispenser having a dispenser pump; b) activating the dispenser pump to combine the composition with air such that the composition foams as it is dispensed using the dispenser pump; and c) applying the foam to the skin. In one embodiment, the composition used in the method contains no additive or surfactant. In another embodiment, the composition contains an additive or a second surfactant. In an alternate embodiment, the composition contains an additive and a second surfactant.

The disclosure further provides is an unpressurized dispenser having a dispenser pump for mixing a composition with air and dispensing foam, the dispenser storing a foamable composition comprising: a short-chain alcohol or mixtures thereof, a surfactant, 7.5% weight or more of at least one sensation modifying agent, optionally, an additive and optionally, a second surfactant. wherein the composition is forced through a fine mesh screen and mixed with air at low pressure to form a foam.

The disclosure further provides a method of forming a foam using the unpressurized dispenser, the method comprising activating the dispenser pump to mix a foamable composition disclosed above with air through a fine mesh screen under low pressure conditions to form a foam which is dispensed from said dispenser.

The disclosure further provides a method of producing and dispensing a foam using a foamable composition, the method comprising: dispensing a foamable composition from a container having a dispenser pump configured to mix air through a fine mesh screen with the foamable composition disclosed above under low pressure during dispensing to form a foam, wherein the foamable composition comprising a short-chain alcohol or mixtures thereof, a surfactant, 7.5% weight or more of at least one sensation modifying agent, optionally, an additive and optionally, a second surfactant.

The disclosure further provides a process for the preparation of a foam composition comprising the following steps:
(i) preparing a mixture of a short-chain alcohol or mixtures thereof with a surfactant;
(ii) adding to the mixture above 7.5% weight or more of at least one sensation modifying agent;
(iii) optionally adding to the mixture above an additive and/or a second surfactant; and
(iv) adding water.

The examples shown hereinbelow are intended to exemplify some of the ways of carrying out the invention, however without limiting its scope.

### EXAMPLES

### Example 1—Foaming Composition and Its Surfactant

The base formula considered in this example according to Table 1 below.

TABLE 1
Base formula

| Ingredient | Quantity |
|---|---|
| SD-Alcohol | 50% |
| Water | 24% |
| Menthol | 16% |
| Surfactant | 10% |

Several surfactants were tested and evaluated according to the foam quality. The results are presented in Table 2 below.

Table 2 shows multiple foam composition embodiments comprising different surfactants in the base formula of Table 1 using a 200/100 mesh with a 1.5 mL output. Multiple surfactants with the same INCI name from different manufacturers and different molecular weights were tested in comparison, with similar results.

TABLE 2
Multiple foam composition embodiments comprising different
surfactants in the base formula of Table 1

| INCI | Evaluation |
|---|---|
| Sodium Lauryl Sulfate | No foam |
| Ammonium Lauryl Sulfate | No foam |
| Cocamidopropyl Betaine | No foam |
| Sodium Lauryl GL. Carb (and) Lauryl | No foam |
| Glucoside | |
| PEG-12-Dimethicone | Some foam |
| PEG-40 Glyceryl Cocoate & Sodium | No foam |
| Coceth Sulfate | |
| Caprylrl/Capryl Glucoside | No foam |
| Sodium Lauroyl Sarcosinate | No foam |
| Sodium Lauroyl Oat Amino Acids | No foam |
| Sodium Cocoyl Apple Amino Acids | No foam |
| C15-19 Alkane | No foam |

TABLE 2
Multiple foam composition embodiments comprising different
surfactants in the base formula of Table 1

| INCI | Evaluation |
|---|---|
| Decyl Glucoside & Cocamidopropyl | No foam |
| Betaine | |
| Decyl Glucoside | No foam |
| Sodium Lauroamphoacetate | No foam |
| PEG-8 PG Coco-Glucoside Dimethicone | Some foam |
| Hydoxypropyl Bis-Hydroxyethyldimonium | No foam |
| Chloride | |
| Cocamidopropyl Hydroxysultaine | No foam |
| Sorbitan Oleate Decylglucoside | No foam |
| Crosspolymer | |
| bis-Stearyl Dimethicone | Some foam |
| PEG-10 Dimethicone | Poor foam |
| PEG-8 Dimethicone | Poor foam |
| bis-PEG-12 Dimethicone** | Good foam |
| bis-PEG-10 Dimethicone** | Good foam |
| bis-PEG-8 Dimethicone** | Good foam |
| bis-PEG/PPG-14/14 Dimethicone | Some foam |
| PEG/PPG-18/18 Dimethicone | Some foam |

The results of Table 2 above show good foam quality in those embodiments comprising bis-PEG dimethicones as surfactant. The embodiments comprising Bis-PEG/PPG-14/14 dimethicone and PEG/PPG-18/18 dimethicone as surfactant also provided good foam quality. **For the bis-PEG dimethicones herein described, a composition containing 10.0% of menthol was also tested. The foam quality further improved.

### Example 2—Foaming Composition and Surfactant Combinations

The base formula considered in this example according to Table 3 below.

TABLE 3
Base formula containing two surfactants

| Ingredient | Quantity |
|---|---|
| SD-Alcohol | 50% |
| Water | 24% |
| Menthol | 16% |
| Surfactant 1 | 5% |
| Surfactant 2 | 5% |

Several surfactant combinations were tested and evaluated according to the foam quality. The results are presented in Table 4 below.

Table 4 shows multiple foam composition embodiments comprising different surfactant combinations in the base formula of Table 3 using a 200/100 mesh with a 1.5 mL output. Multiple surfactants with same INCI name from different manufacturers and different molecular weights were tested in comparison, with similar results.

TABLE 4
Multiple foam composition embodiments comprising different
surfactant combinations in the base formula of Table 3

| Surfactant 1 (INCI Name) | Surfactant 2 (INCI Name) | Result |
|---|---|---|
| bis-PEG-12 Dimethicone | bis-PEG-10 Dimethicone | Good foam |
| bis-PEG-12 Dimethicone | bis-PEG-8 Dimethicone | Good foam |
| bis-PEG-12 Dimethicone | PEG-12 Dimethicone | Good foam |
| bis-PEG-12 Dimethicone | bis-PEG/PPG-14/14 | Good foam |
| | Dimethicone | |
| bis-PEG-12 Dimethicone | PEG/PPG-18/18 | Good foam |
| | Dimethicone | |
| bis-PEG-10 Dimethicone | bis-PEG-8 Dimethicone | Good foam |
| bis-PEG-10 Dimethicone | bis-PEG/PPG-14/14 | Good foam |
| | Dimethicone | |
| bis-PEG-10 Dimethicone | PEG/PPG-18/18 | Good foam |
| | Dimethicone | |
| bis-PEG-8 Dimethicone | bis-PEG/PPG-14/14 | Some foam |
| | Dimethicone | |
| bis-PEG-8 Dimethicone | PEG/PPG-18/18 | Non-uniform |
| | Dimethicone | foam |
| PEG-12 Dimethicone | bis-PEG/PPG-14/14 | Good foam |
| | Dimethicone | |
| PEG-12 Dimethicone | PEG/PPG-18/18 | Good foam |
| | Dimethicone | |
| bis-PEG/PPG-14/14 | PEG/PPG-18/18 | Poor quality |
| Dimethicone | Dimethicone | foam |

The results of this evaluation show that all foam composition embodiments comprising two surfactants provided usable foams with increased residence time.

### Example 3—Foaming Composition and Alcohol Concentrations

In one embodiment, the concentration range of alcohol used for the base formula was evaluated. The base formula used in this embodiment is that of Table 5 below.

TABLE 5
Base formula

| Ingredient | Quantity |
|---|---|
| SD-Alcohol | More than 30% |
| Water | Less than 44% |
| Menthol | 16% |
| Surfactant | 10% |

Table 6 shows multiple foam composition embodiments comprising different alcohol concentrations in the base formula of Table 5 using a 200/100 mesh with 1.5 mL output.

TABLE 6
Multiple foam composition embodiments comprising different
alcohol concentrations in the base formula of Table 5

| % SD Alcohol | % H₂O | Results |
|---|---|---|
| 30 | 44 | Poor quality foam |
| 35 | 39 | Poor quality foam |
| 40 | 34 | Good foam |
| 50 | 24 | Good foam |
| 60 | 14 | Good foam |

The results of Table 6 above show that 40-60% alcohol gave good results.

### Example 4—Foaming Composition with Optional Additives

The foaming composition of the invention may optionally comprise further additives. In this example, multiple foam composition embodiments with the base composition of Table 7 were tested with different additives.

TABLE 7
Base formula

| Ingredient | Quantity |
|---|---|
| SD-Alcohol | 50% |
| Water | 19% |
| Menthol | 16% |
| Surfactant bis-PEG-10 Dimethicone | 10% |
| Additive | 5% |

The additives themselves are not primarily intended to improve foaming properties. While additives were tested for enhancing other properties such as moisturization and feel on skin, their effect on foaming properties was also evaluated.

Table 8 shows multiple foam composition embodiments comprising different additives in the base composition of Table 7 using a 200/100 mesh with a 1.5 mL output.

TABLE 8
Multiple foam composition embodiments comprising different
additives in the base composition of Table 7

| INCI | Evaluation |
|---|---|
| Distearyldimonium Chloride | Took 4 hours to dissolve |
| Ethoxydiglycol | Good foam |
| PPG12/SMDI Copolymer | Non-uniform bubbles |
| PPG-Benzyl Ether Myristate | Non-uniform bubbles |
| B-LC/HDI/PEG-100 Copolymer | Non-uniform bubbles |
| Polysorbate 60 | Good foam |
| Cetyl Dimethicone | Non-uniform bubbles |
| Acrylates/C10-30 Alkyl Acrylate | Good foam |
| Crosspolymer | |
| PEG-3 Dioleoylamidoethylmonium | Good foam |
| Methyl Gluceth-20 | Good foam |
| Polyquaternium-67 | Clogged foamer pump |

TABLE 8
Multiple foam composition embodiments comprising different
additives in the base composition of Table 7

| INCI | Evaluation |
|---|---|
| PEG-14M | Clogged foamer pump |
| Propylene Glycol | Good foam |
| Cocamidopropyl Betaine | Non-uniform foam |
| PEG-12-Dimethicone | Good foam |
| Dimethicone 20 Cosmetic | No foam |
| Dimethicone Crosspolymer Blend | No foam |
| Glycerin 99% Kosher USP | Less foam than base |
| Diisopropyl Adipate | Good foam |
| Ethylhexylglycerin | Good foam |
| Isopropyl Myristate | Good foam |
| HPMC 4000 | Clogged foamer pump |
| Behentrimonium Methosulfate | Good foam |

The results show that the additives which provided similar foaming properties as those of the base composition base are selected from: ethoxydiglycol, polysorbate 60, acrylates/C10-30 alkyl acrylate crosspolymer, PEG-3 dioleoylamidoethylmonium, methyl gluceth-20, propylene glycol, PEG-12-dimethicone, diisopropyl adipate, ethylhexylglycerin, isopropyl myristate, behentrimonium methosulfate, glycerin 99% kosher USP. Also, the foam composition embodiment comprising propylene glycol showed improved moisturization while retaining the foam properties of the base composition of Table 7.

### Example 5—Foaming Compositions with Different Propylene Glycol Concentrations

In this example, a set of experiments was designed to evaluate the effect of different concentrations of propylene glycol in the foam composition.

The base formula of Table 9 was used in multiple foam composition embodiments comprising different concentrations of propylene glycol.

TABLE 9
Base formula

| Ingredient | Quantity |
|---|---|
| SD-Alcohol | 50% |
| Menthol | 16% |
| Water | More than 14% |
| bis-PEG-10 Dimethicone | 10% |
| Propylene Glycol | Less than 10% |

Table 10 shows multiple foam composition embodiments comprising different concentrations of propylene glycol in the base composition of Table 9, and their results of foam formation comparing the foaming with 5% propylene glycol; using a 200/100 mesh with a 1.5 mL output.

TABLE 10
Multiple foam composition comprising different concentrations
of propylene glycol in the base composition of Table 9

| % Propylene Glycol | % Water | Results |
|---|---|---|
| 10 | 14 | Decreased foam vs 5% |
| 7.5 | 16.5 | Decreased foam vs 5% |
| 5 | 19 | Good foam |
| 4 | 20 | Good foam |
| 3 | 21 | Good foam |
| 2.5 | 21.5 | Good foam |
| 1 | 23 | Good foam |
| 0.5 | 23.5 | Good foam |

The results show that propylene glycol concentrations over 5% tend to decrease foaming properties, and 0.5% to 5% propylene glycol provide good results.

### Example 6—Foaming Compositions Comprising Two Different Surfactants

To analyze the impact of the surfactant ratio in the foaming properties of compositions comprising two surfactants, the following experiment was conducted. A base foam composition according to Table 11 was used.

TABLE 11
Base formula

| Ingredient | Quantity |
|---|---|
| SD-Alcohol | 50% |
| Water | More than 24% |
| Menthol | 16% |
| Bis-PEG-12 Dimethicone | Less than 6% |
| Bis-PEG-8 Dimethicone | Less than 6% |

Multiple foam composition embodiments comprising the base composition of Table 11 and comprising two surfactants with different concentrations were analyzed. The results are shown in Table 12.

Table 12 shows the foaming properties of multiple foam composition embodiments comprising the base composition of Table 11 and comprising two surfactants with different concentrations using a 200/100 mesh with a 1.5 mL output.

TABLE 12
Foaming properties of multiple foam composition comprising
the base composition of Table 11 and two different surfactants

| | | | | | | Result |
|---|---|---|---|---|---|---|
| | | | | | | (foam |
| | | | | | | quality |
| | | | | | | compared |
| | | | | | % | to |
| Test | Surfactant 1 | % | Surfactant 2 | % | water | baaseline) |
| Ratio | bis-PEG-12 | 5 | bis-PEG-8 | 5 | 24 | Baseline |
| | Dimethicone | | Dimethicone | | | |
| | bis-PEG-12 | 4 | bis-PEG-8 | 6 | 24 | Better |
| | Dimethicone | | Dimethicone | | | |
| | bis-PEG-12 | 6 | bis-PEG-8 | 4 | 24 | Worse |
| | Dimethicone | | Dimethicone | | | |
| Total % | bis-PEG-12 | 4 | bis-PEG-8 | 4 | 26 | No change |
| | Dimethicone | | Dimethicone | | | |
| | bis-PEG-12 | 3 | bis-PEG-8 | 3 | 28 | No change |
| | Dimethicone | | Dimethicone | | | |
| | bis-PEG-12 | 2.5 | bis-PEG-8 | 2.5 | 29 | No change |
| | Dimethicone | | Dimethicone | | | |
| | bis-PEG-12 | 2 | bis-PEG-8 | 2 | 30 | Worse |
| | Dimethicone | | Dimethicone | | | |
| | bis-PEG-12 | 1 | bis-PEG-8 | 1 | 32 | Worse |
| | Dimethicone | | Dimethicone | | | |
| Ratio with | bis-PEG-12 | 3 | bis-PEG-8 | 2 | 29 | Worse |
| decreased | Dimethicone | | Dimethicone | | | |
| % | bis-PEG-12 | 2 | bis-PEG-8 | 3 | 29 | Better |
| | Dimethicone | | Dimethicone | | | |
| | bis-PEG-12 | 1.5 | bis-PEG-8 | 3.5 | 29 | Better |
| | Dimethicone | | Dimethicone | | | |
| | bis-PEG-12 | 1 | bis-PEG-8 | 4 | 29 | Worse |
| | Dimethicone | | Dimethicone | | | |
| | bis-PEG-12 | 0.5 | bis-PEG-8 | 4.5 | 29 | Worse |
| | Dimethicone | | Dimethicone | | | |

The results show that as for the ratio, using higher amounts of bis-PEG-8 dimethicone than bis-PEG-12-dimethicone results in better foaming. The results of total % of surfactants show that 5% of total surfactants suffices for good foaming. Also, using surfactants with dief a ratio of bis-PEG-12 dimethicone to bis-PEG-8 dimethicone in the range 1.5 to 2.33 provides better results, that is, 4:6, 2:3 and 1.5:3.5.

### Example 7—Foaming Tests for Assessing Foam Quality

Foaming quality assessments were performed using different actuators, mesh sizes and stroke volumes. in these experiments, stroke volume refers to the amount of air being infused into the liquid.

TABLE 13
Foaming tests with composition comprising 10% menthol

| Stroke Volume (mL) | Mesh Size | Actuator Head | Result |
|---|---|---|---|
| 1.2 | “Velvet” | Square | Non-uniform foam |
| 1.5 | “Velvet” | Square | Non-uniform foam |
| 1.2 | 80/70 | Square | Poor quality foam |
| 1.5 | 80/70 | Square | Poor quality foam |
| 1.2 | 200/100 | Square | Good foam |
| 1.5 | 200/100 | Square | Good foam |
| 1.2 | 222/150 | Square | Non-uniform foam |
| 1.5 | 222/150 | Square | Non-uniform foam |

TABLE 14
Foaming tests with composition comprising 16% menthol

| Stroke Volume (mL) | Mesh Size | Actuator Head | Result |
|---|---|---|---|
| 1.2 | “Velvet” | Square | Some foam |
| 1.5 | “Velvet” | Square | No foam |
| 1.2 | 80/70 | Square | Poor quality foam |
| 1.5 | 80/70 | Square | Poor quality foam |
| 1.2 | 200/100 | Square | Good foam |
| 1.5 | 200/100 | Square | Good foam |
| 1.2 | 222/150 | Square | Non-uniform foam |
| 1.5 | 222/150 | Square | Non-uniform foam |
| 0.8 | 200/100 | Foghorn | Good foam |
| 1.2 | 200/100 | Foghorn | Good foam |
| 0.8 | 150/250 | Foghorn | Good foam |
| 1.2 | 150/250 | Foghorn | Good foam |
| 0.8 | 100/300 | Foghorn | No foam |
| 1.2 | 100/300 | Foghorn | No foam |

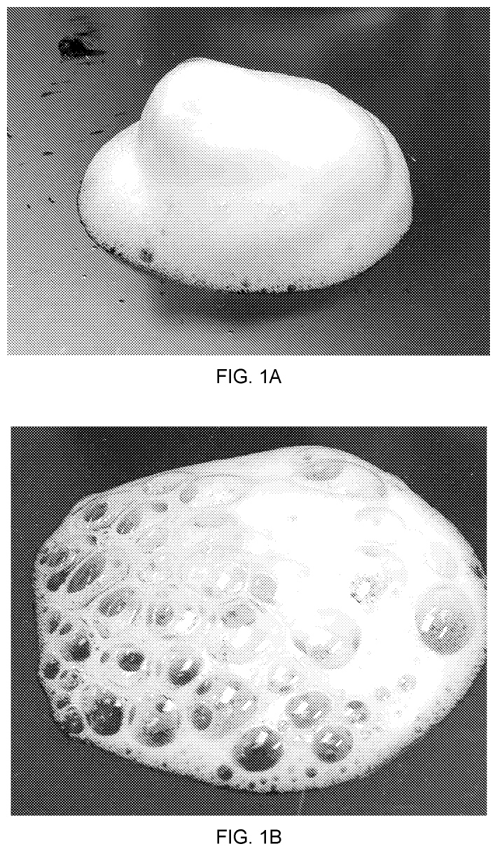

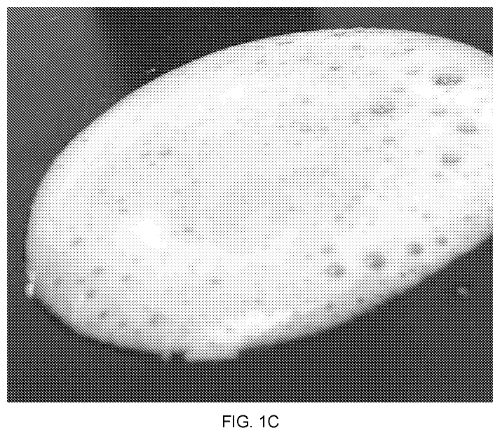

Tables 13 and 14 above show that the foam evaluation for different formulas with air infusing “foaming” pump actuators, with different mesh screen sizes and stroke volumes, which were visually inspected. Results with 200/100 and 150/250 mesh provided best foam; stroke volumes of 0.8 mL, 1.2 mL, and 1.5 mL provided similar good results, but increased output amount occurred with increased stroke volume. 0.8 mL stroke volume was chosen to optimize output for single site of administration. Results were independent of actuator head as mesh placed into different shaped actuator resulted in similar quality foam. FIG. 1A shows example of good foam with good height and uniform bubbles, which did not collapse immediately. FIG. 1B and FIG. 1C show examples of non-uniform foam and poor quality foam, respectively.

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention, Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### Numbered Embodiments of the Invention

Provided here are numbered embodiments of the disclosed technology. These embodiments are illustrative only and do not limit the scope of the present disclosure or of the claims attached.

Embodiment 1. A foamable composition comprising: (i) a short-chain alcohol or mixtures thereof; (ii) a surfactant; (iii) 7.5% weight or more of at least one sensation modifying agent; (iii) water; and, optionally, (iv) an additive and/or a second surfactant.

Embodiment 2. The composition of embodiment 1, wherein the short-chain alcohol is selected from C₁-C₆ alkyl alcohol or combinations thereof.

Embodiment 3. The foamable composition of embodiment 2, wherein the short-chain alcohol is ethanol.

Embodiment 4. The foamable composition of any one of embodiments 1-3, wherein the short-chain alcohol is present in an amount in the range of 35.0% to 60.0% by weight of the total composition.

Embodiment 5. The foamable composition of any one of embodiments 1-4, wherein said surfactant is a dimethicone-based surfactant.

Embodiment 6. The foamable composition of embodiment 5, wherein said dimethicone-based surfactant is selected from the group consisting of: PEG-8 PG coco-glucoside dimethicone, bis-stearyl dimethicone, PEG-8 dimethicone, PEG-12 dimethicone, bis-PEG-12 dimethicone, bis-PEG-10 dimethicone, bis-PEG-8 dimethicone, bis-PEG/PPG-14/14 dimethicone, or PEG/PPG-18/18 dimethicone.

Embodiment 7. The foamable composition of embodiment 6, wherein the dimethicone-based surfactant is selected from the group consisting of bis-PEG-12 dimethicone and bis-PEG-8 dimethicone.

Embodiment 8. The foamable composition of any one of embodiments 1-7, wherein the surfactant is present in an amount of at least 0.1% by weight of the total composition.

Embodiment 9. The foamable composition of embodiment 8, wherein the surfactant is present in an amount between about 2.0% to about 10.0% by weight of the total composition.

Embodiment 10. The foamable composition of any one of embodiments 1-9, wherein the sensation modifying agent is selected from the group consisting of: a cooling agent; a warming agent; a relaxing or soothing agent; stimulating or refreshing agent; or mixtures thereof.

Embodiment 11. The foamable composition of embodiment 10, wherein the sensation modifying agent comprises capsaicin and/or methyl salicylate.

Embodiment 12. The foamable composition of embodiment 10, wherein the cooling agent is selected from camphor or camphor derivative; menthol; an isomer of menthol, a menthol derivative; 4-methyl-3-(1-pyrrolidinyl)-2[5H]-furanone; WS-23, Icilin, Icilin Unilever Analog, 5-methyl-4-(1-pyrrolidinyl)-3-[2H]-furanone; 4,5-dimethyl-3-(1-pyrrolidinyl)-2[5H]-furanone, isopulegol, 3-(l-menthoxy)propane-1,2-diol, 3-(l-menthoxy)-2-methylpropane-1,2-diol, p-menthane-2,3-diol, p-menthane-3,8-diol, 6-isopropyl-9-methyl-1,4-dioxas-piro[4,5]decane-2-methanol, menthyl succinate and its alkaline earth metal salts, trimethylcyclohexanol, N-ethyl-2-isopropyl-5-methylcyclohexanecarb-oxamide, Japanese mint (Mentha arvensis) oil, peppermint oil, menthone, menthone glycerol ketal, menthyl lactate, 3-(l-menthoxy)ethan-1-ol, 3-(l-menthoxy)propan-1-ol, 3-(l-menthoxy)butan-1-ol, l-menthylacetic acid N-ethylamide, l-menthyl-4-hydroxypentanoate, l-menthyl-3-hydroxybutyrate, N,2,3-trimethyl-2-(1-methylethyl)-butanamide, and spearmint oil.

Embodiment 13. The foamable composition of embodiment 12, wherein the cooling agent is present in an amount of between 7.5% and 16.0% by weight of the total composition.

Embodiment 14. The foamable composition of any one of embodiments 1-13, wherein the additive is selected from the group consisting of: ethoxydiglycol, polysorbate 60, acrylates/C10-30 alkyl acrylate crosspolymer, PEG-3 dioleoylamidoethylmonium, methyl gluceth-20, propylene glycol, PEG-12-dimethicone, diisopropyl adipate, ethylhexylglycerin, isopropyl myristate, behentrimonium methosulfate, and glycerin 99% kosher USP.

Embodiment 15. The foamable composition of embodiment 14, wherein the additive is propylene glycol.

Embodiment 16. The foamable composition of embodiment 14, wherein the additive is presented in an amount of equal to or less than 5.0% by weight of the total composition.

Embodiment 17. The foamable composition of any one of embodiments 1-16, wherein said second surfactant is a dimethicone-based surfactant.

Embodiment 18. The foamable composition of embodiment 17, wherein said second surfactant is present in an amount of at least 0.1% by weight of the total composition.

Embodiment 19. The foamable composition of embodiment 17, wherein the two acceptable surfactants are bis-PEG-12 dimethicone and bis-PEG-8 dimethicone.

Embodiment 20. The foamable composition of embodiment 19, wherein the percentages of bis-PEG-12 Dimethicone to bis-PEG-8 Dimethicone is in the range 1.0 to 3.5, respectively.

Embodiment 21.A foam composition comprising: (i) 35.0% to 65.0% of SD-alcohol; (ii) 1.0% to 10.0% of a dimethicone surfactant; (iii) 7.5% to 16.0% of menthol; (iv) water; and, optionally, (v) 0.5% to 5.0% of an additive and/or 0.5% to 6.0% of a second dimethicone surfactant.

Embodiment 22.A foam composition comprising: (i) 45.0% to 65.0% of SD-alcohol; (ii) 1.5% to 4.0% of a bis-PEG-12 dimethicone; (iii) 3.5% to 6.0% of bis-PEG-8 dimethicone; (iv) 7.5% to 16.0% of menthol; (v) water; and optionally, (vi) 0.5% to 5.0% of an additive.

Embodiment 23. A process for the preparation of the foam composition of any one of embodiments 1-20 comprising the following steps: (i) preparing a mixture of a short-chain alcohol or mixtures thereof with an acceptable surfactant; (ii) adding to the mixture above 7.5% weight or more of at least one sensation modifying agent; (iii) optionally adding to the mixture above an additive and/or a second acceptable surfactant; and (iv) adding water.

1. A foamable composition comprising:
(i) a short-chain alcohol or mixtures thereof;
(ii) a surfactant;
(iii) 7.5% weight or more of at least one sensation modifying agent;
(iv) water; and, optionally,
(v) an additive and/or a second surfactant.

2. The foamable composition of claim 1, wherein the short-chain alcohol is selected from C₁-C₆ alkyl alcohol or combinations thereof.

3. The foamable composition of claim 2, wherein the short-chain alcohol is ethanol.

4. The foamable composition of claim 1, wherein the short-chain alcohol is present in an amount in the range of 35.0% to 60.0% by weight of the total composition.

5. The foamable composition of claim 1, wherein said surfactant is a dimethicone-based surfactant.

6. The composition of claim 5, wherein said dimethicone-based surfactant is selected from the group consisting of: PEG-8 PG coco-glucoside dimethicone, bis-stearyl dimethicone, PEG-8 dimethicone, PEG-12 dimethicone, bis-PEG-12 dimethicone, bis-PEG-10 dimethicone, bis-PEG-8 dimethicone, bis-PEG/PPG-14/14 dimethicone, or PEG/PPG-18/18 dimethicone.

7. The foamable composition of claim 6, wherein the dimethicone-based surfactant is selected from the group consisting of bis-PEG-12 dimethicone and bis-PEG-8 dimethicone.

8. The foamable composition of claim 1, wherein the surfactant is present in an amount of at least 0.1% by weight of the total composition.

9. The foamable composition of claim 8, wherein the surfactant is present in an amount between about 2.0% to about 10.0% by weight of the total composition.

10. The foamable composition of claim 1, wherein the sensation modifying agent is selected from the group consisting of: a cooling agent; a warming agent; a relaxing or soothing agent; stimulating or refreshing agent; or mixtures thereof.

11. The foamable composition of claim 10, wherein the sensation modifying agent comprises capsaicin and/or methyl salicylate.

12. The foamable composition of claim 10, wherein the cooling agent is selected from camphor or camphor derivative; menthol; an isomer of menthol, a menthol derivative; 4-methyl-3-(1-pyrrolidinyl)-2[5H]-furanone; WS-23, Icilin, Icilin Unilever Analog, 5-methyl-4-(1-pyrrolidinyl)-3-[2H]-furanone; 4,5-dimethyl-3-(1-pyrrolidinyl)-2[5H]-furanone; isopulegol, 3-(l-menthoxy)propane-1,2-diol, 3-(l-menthoxy)-2-methylpropane-1,2-diol, p-menthane-2,3-diol, p-menthane-3,8-diol, 6-isopropyl-9-methyl-1,4-dioxas-piro[4,5]decane-2-methanol, menthyl succinate and its alkaline earth metal salts, trimethylcyclohexanol, N-ethyl-2-isopropyl-5-methylcyclohexanecarb-oxamide, Japanese mint (Mentha arvensis) oil, peppermint oil, menthone, menthone glycerol ketal, menthyl lactate, 3-(l-menthoxy)ethan-1-ol, 3-(l-menthoxy)propan-1-ol, 3-(l-menthoxy)butan-1-ol, l-menthylacetic acid N-ethylamide, l-menthyl-4-hydroxypentanoate, l-menthyl-3-hydroxybutyrate, N,2,3-trimethyl-2-(1-methylethyl)-butanamide, and spearmint oil.

13. The foamable composition of claim 12, wherein the cooling agent is present in an amount of between 7.5% and 16.0% by weight of the total composition.

14. The foamable composition of claim 1, wherein the additive is selected from the group consisting of: ethoxydiglycol, polysorbate 60, acrylates/C10-30 alkyl acrylate crosspolymer, PEG-3 dioleoylamidoethylmonium, methyl gluceth-20, propylene glycol, PEG-12-dimethicone, diisopropyl adipate, ethylhexylglycerin, isopropyl myristate, behentrimonium methosulfate, and glycerin 99% kosher USP.

15. The foamable composition of claim 14, wherein the additive is propylene glycol.

16. The foamable composition of claim 14, wherein the additive is presented in an amount of equal to or less than 5.0% by weight of the total composition.

17. The foamable composition of claim 1, wherein said second surfactant is a dimethicone-based surfactant.

18. The foamable composition of claim 17, wherein said second surfactant is present in an amount of at least 0.1% by weight of the total composition.

19. The foamable composition of claim 17, wherein the two acceptable surfactants are bis-PEG-12 dimethicone and bis-PEG-8 dimethicone.

20. The foamable composition of claim 19, wherein the percentages of bis-PEG-12 Dimethicone to bis-PEG-8 Dimethicone is in the range 1.0 to 3.5, respectively.

21. A foam composition comprising:
(i) 35.0% to 65.0% of SD-alcohol;
(ii) 1.0% to 10.0% of a dimethicone surfactant;
(iii) 7.5% to 16.0% of menthol;
(iv) water; and, optionally,
(v) 0.5% to 5.0% of an additive and/or 0.5% to 6.0% of a second dimethicone surfactant.

22. A foam composition comprising:
(i) 45.0% to 65.0% of SD-alcohol;
(ii) 1.5% to 4.0% of a bis-PEG-12 dimethicone;
(iii) 3.5% to 6.0% of bis-PEG-8 dimethicone;
(iv) 7.5% to 16.0% of menthol;
(v) water; and optionally,
(vi) 0.5% to 5.0% of an additive.

23. A process for the preparation of the foam composition of claim 1 comprising the following steps:
(i) preparing a mixture of a short-chain alcohol or mixtures thereof with an acceptable surfactant;
(ii) adding to the mixture above 7.5% weight or more of at least one sensation modifying agent;
(iii) optionally adding to the mixture above an additive and/or a second acceptable surfactant; and
(iv) adding water.
